(19) United States
(12) Patent Application Publication
(10) Pub. No.: US 2015/0238261 A1
(43) Pub. Date: Aug. 27, 2015

(54) SYSTEM AND METHOD FOR TREATMENT USING A LASER BEAM DELIVERED VIA AN OPTICAL FIBER

(71) Applicant: Convergent Dental, Inc., Natick, MA (US)

(72) Inventors: Nathan P. Monty, Shrewsbury, MA (US); Charles H. Dresser, Bethel, ME (US)

(21) Appl. No.: 14/634,284

(22) Filed: Feb. 27, 2015

Related U.S. Application Data
(60) Provisional application No. 61/945,633, filed on Feb. 27, 2014.

(51) Int. Cl. A61B 18/22

(52) U.S. Cl. CPC A61B 18/22; A61B 2018/00017

(57) ABSTRACT

A laser system can efficiently direct a laser beam to a treatment area via an optical fiber. The laser beam is produced using a CO₂ laser that has a wavelength greater than about 9 μm and less than about 10 μm. The optical fiber, which can be a solid core fiber, includes chalcogenide glass. The chalcogenide glass is adapted to minimize transmission losses at wavelengths in a range from about 9 μm up to about 10 μm. One or more laser beam parameters can be controlled at least according to a selected treatment using a controller. A visible-spectrum and/or fluorescing optical fiber may be physically but not optically coupled to the chalcogenide fiber.

### CROSS-REFERENCE TO RELATED APPLICATIONS

This application claims benefit of priority to U.S. Provisional Patent Application No. 61/945,633, entitled “System And Method For Treatment Using A Laser Beam Delivered Via An Optical Fiber,” filed on Feb. 27, 2014, the contents of which are incorporated herein by reference in their entirety.

### FIELD OF THE INVENTION

This disclosure generally relates to dental and surgical treatment of tissue using a laser beam and, in particular, to delivering laser radiation to a treatment region, via an optical fiber.

### BACKGROUND

Laser radiation at wavelengths in about 9-10 μm range is well absorbed by many biological substances, including hydroxyapatite in hard tissue, and water and collagen in soft tissue. The use of this wavelength range has also been studied for the ablation of dental hard tissue. Enamel, for example, is primarily hydroxyapatite and, as such, may be ablated using a laser emitting radiation at wavelengths in about 9-10 μm range (referred to as a 9-10 μm laser). A 9.3 μm CO₂ laser has recently gained approval for cavity preparation by the U.S. Food and Drug Administration (FDA). A 10.6 μm infrared CO₂ laser may be used for incision, excision, and ablation of soft tissue, at least in part because the 10.6 μm wavelength is well absorbed by water and collagen. The absorption characteristics of water and collagen at the 10.6 μm wavelength are similar to the water and collagen absorption characteristics at the 9.3 μm wavelength. Therefore, 9-10 μm lasers may also be used for soft tissue treatment.

Various 9-10 μm lasers may be used for treatment for prevention of cavities in dental enamel and root surfaces. Caries typically form when acids, the byproduct of sugar consumed by bacteria in the mouth, weaken the tooth tissue. The presence of carbonate in the tooth structure can play an important role in the progression of caries in teeth. Carbonate from tooth surfaces can be removed when the surface temperature of the tooth is increased momentarily, e.g., for 2 μs, 10 μs, 100 μs, 1000 us, 0.5 s. Laser radiation at a power setting below the ablation threshold of hard dental tissue can remove carbonate molecules from the tooth surface. The resulting de-carbonated tooth surfaces may be 75%-80% more resistant to the progression of caries relative to untreated, carbonated tooth surfaces. The 9-10 μm lasers are well suited for this purpose, in part because the radiation at these wavelengths can efficiently increase the surface temperature of the tooth, without significant unintended heating of tissue below the surface. The radiation at wavelengths outside the 9-10 μm range is not absorbed by hard tissue as effectively as 9-10 μm radiation is. Therefore, many lasers emitting at wavelengths outside the 9-10 μm range are not well suited for treatment of hard tissue.

In the past, the only lasers emitting radiation at wavelengths in the range 9-10 μm with enough power to ablate hard and/or soft tissue were TEA lasers. These lasers are generally very costly and, hence, have not been widely used for dental and/or surgical treatment. Recently, however, a CO₂ laser was developed using isotopic oxygen with a molecular mass of 18 g/mol, so as to produce emission at wavelength of 9.3 μm. Systems and methods described in U.S. Pat. No. 8,251,984, the disclosure of which is incorporated herein in its entirety, have facilitated the production of 9-10 μm lasers with power sufficient to make widespread clinical use of radiation at wavelengths in the desirable 9-10 μm range practical.

In addition to their high degree of absorption in hard and soft dental tissue, CO₂ lasers emitting radiation at wavelengths in the range of about 9-10 μm have numerous other clinical benefits. For example, such CO₂ lasers can be operated with pulse durations on the order of 10 s of microseconds. Other near and mid-infrared lasers, such as Erbium YAG lasers, have a pulse duration on the order of 100 s of microseconds. The shorter pulse duration of the CO₂ lasers allows for delivering relatively less power per pulse to the tissue to be treated using the CO₂ laser than using other lasers, such Er:YAG lasers. The shorter pulse durations are beneficial in dental and medical applications, because the shorter pulse widths can limit the thermal damage to the tissue that is not to be treated and is adjacent to the tissue to be irradiated by the laser. CO₂ lasers can also deliver laser pulses at a relatively high pulse repetition rates, often in excess of 10 kHz. Using a train of pulses with a high repetition rate relative to other lasers, a high material removal rate can be achieved, even though, relative to other lasers, only a small amount of material is removed in a single pulse of a CO₂ laser. Thus, the speed at which a 9-10 μm CO₂ laser can remove hard and soft tissue can be fast enough for practical clinical applications. The delivery of such laser radiation, i.e., a laser beam, to the treatment region, however, can be challenging.

Radiation from lasers operating in the near-infrared and mid-infrared wavelength range is often transmitted through fiber optic beam delivery systems, including those having optical fibers based on materials such as Silica, Fluoride doped Silica, Indium Fluoride, and Zirconium Fluoride. Optical fibers of these materials can generally transmit radiation at wavelengths in the range from about 1 μm up to about 4 μm effectively. Therefore, laser diodes and Nd:YAG lasers that operate at a wavelength of about 1 μm can be coupled to an optical fiber based on the materials listed above, and can be used in clinical procedures for soft tissue incision, excision and ablation. Er:YSGG lasers, which operate at a wavelength of 2.8 μm have also been used clinically with a fiber delivery system, to ablate dental hard tissue. Fiber delivery systems have permitted the use of Er:YSGG lasers in endodontic procedures.

These near and mid infrared lasers (e.g., lasers emitting radiation at wavelength in the 1-4 μm range), however, have not seen widespread use in dental and medical applications, in part because of their relatively short wavelengths. The shorter wavelengths of the near and mid-infrared lasers correspond to a longer optical penetration depth in many biological substances. As such, the use of these lasers can cause damage to healthy tissue that is below or adjacent to the treatment region, and that is not to be treated. The CO₂ lasers generally have lower optical penetration depths, mitigating or avoiding the risk of damaging tissue that is not to be treated.

An important limitation of many lasers operating in the far-infrared wavelength spectrum (e.g., 6-15 μm), including the CO₂ lasers, is the lack of a clinically viable fiber optic beam delivery system. Hollow wave guides can be used to deliver radiation form some CO₂ laser systems operating at 10.6 μm. These delivery systems typically lose approximately a quarter to a third of the input laser power within the hollow wave guide. The high loss percentage of hollow wave guides generally precludes their use at the higher power settings that are required to ablate hard tissue. Articulating arms with a multitude of rotating joints and mirrors have been used with some success with lasers operating in the mid and far-infrared range. The mechanical nature of the articulating arm, however, makes its use cumbersome to the clinician. Alignment reliability can also be difficult to achieve with an articulating arm beam delivery system, adversely affecting the accuracy of laser beam delivery.

Silver halide fibers can transmit radiation at wavelengths in the 9-10 μm range, but they are generally not clinically viable for various reasons. For example, a silver halide fiber is a polycrystalline structure and not a glass. As the silver halide fiber is used over time, the number of crystals in the fiber can increase, decreasing the transmission of the fiber over time. Silver halide can also deteriorate in light, causing decreases in transmission capability of the fiber over time. In addition, silver halide fibers tend to be mechanically fragile and are generally prone to breaking in a clinical environment.

Some chalcogenide fibers (i.e., fibers based on one or more of Sulfur (S), Arsenic (As), and Selenium (Se)), can transmit radiation in the far-infrared range, including the 9-10 μm range and at wavelengths beyond that range, e.g., at 10.6 μm, 15 μm, etc. Many chalcogenide glass fibers, however, cause transmission losses between 60%-80% at these wavelengths. The manufacturing of chalcogenide glass fibers usually includes the use of glass ampoules. During a homogenizing process, the ampoule and chalcogenide glass materials are typically heated to a temperature around 750° C. This can cause silicon (Si) or other contaminants in the glass ampoule to migrate into the chalcogenide glass, resulting in contaminants in the fiber. These contaminants typically absorb the far-infrared radiation and may cause much of the losses in chalcogenide optical fibers. U.S. Pat. No. 7,418,835, the disclosure of which is incorporated herein by reference in its entirety, appears to describe a process for making a chalcogenide fiber with a reduced number of contaminants in the fiber. As—Se chalcogenide fibers have been used for delivering radiation from a continuous wave (CW) CO₂ laser at 4 mm and 10 mm wavelengths.

Arsenic-selenium-tellurium (As—Se—Te) glass chalcogenide fiber has been tested for delivering the radiation of a 9.3 μm wavelength CO₂ laser. The As—Se—Te glass fiber generally experiences a change in refractive index with a change in temperature, and the change in the refractive index can produce a thermal lensing of the fiber. Even a low absorption of radiation at the 9.3 μm wavelength by the As—Se—Te glass, the chalcogenide fiber based on this material can heat up as a result of the radiation absorbed therein. The temperature change as the fiber heats up can result in thermal lensing and in the eventual destruction of the (As—Se—Te) chalcogenide fiber.

The use of lasers in a clinical procedure generally requires accurately targeting a specific region of the tissue to be treated. To this end, an aiming or pilot laser emitting radiation in the visible spectrum, that can be seen by an operator, may be used. Hollow wave guides and optical fibers that do not use chalcogenide glass can usually transmit both the treatment beam and the aiming laser beam through the same beam delivery system. As described above, these beam delivery systems generally do not transmit far-infrared laser radiation effectively. While some chalcogenide fibers can deliver near and far infrared radiation (e.g., radiation at wavelengths in the 6-15 μm range), such chalcogenide fibers are generally unable to pass light in the visible spectrum, limiting their use in many clinical procedures.

In summary, CO₂ lasers operating at a wavelength of about 9.3 μm, and other 9-10 μm lasers, may be well suited for the treatment (e.g., ablation, prevention of dental caries, incisions, etc.), of both hard and soft tissue. The clinical use of 9-10 μm lasers is limited, however, due to a lack of a suitable, efficient beam delivery system. Specifically, wave guides and articulating arms can be cumbersome and lossy; several optical fibers do not transmit radiation at wavelengths in the 9-10 μm range; typical chalcogenide fiber based beam delivery systems may transmit radiation at wavelengths greater than 10 μm (e.g., 10.6 μm), and at those wavelengths and at wavelengths within the 9-10 μm range, many chalcogenide fibers generally introduce transmission losses and/or losses due to a change in refractive index due to a change in temperature.

Improved systems and methods are therefore needed for efficient delivery of laser radiation in about the 9-10 μm wavelength range.

### SUMMARY

In various embodiments, a laser beam having a wavelength in the range of about 9-10 μm can be delivered efficiently using an optical fiber coupled to the source of the laser beam. This is achieved, in part, by using a solid core chalcogenide fiber that is customized for transmitting radiation in a wavelength range of about 9 μm up to about 10 μm. As used in this disclosure, about means within a tolerance of less than 0.2%, 0.5%, 1%, 2%, 5%, 10%, etc. Such a fiber may include chalcogenide glass that includes arsenic and selenium, and is further characterized by an absence of at least one of tellurium and germanium, or both. The presence of one or more of these compounds may be desirable for the transmission of a wavelength greater than 10 μm, but these compounds can cause adverse effects during transmission of the radiation of the 9-10 μm lasers.

The chalcogenide fiber may be physically, but not optically, coupled to at least one other optical fiber that includes silica and/or a fluoride. Such a fiber can transmit radiation in the visible spectrum for marking and/or radiation that can fluoresce depending on the nature of the target tissue, enabling diagnostics of such tissue. The system may also include a laser controller than can adjust one or more parameters of the laser beam according to the type of the treatment selected and/or the type of the tissue being treated. During treatment, the laser beam may be directed to the treatment area, which may include hard and/or soft tissue, through a medium that does not substantially absorb the radiation, allowing for delivery of a specified energy profile at or near the treatment area. Such a medium can include a gas (e.g., air, nitrogen) and/or a mist.

Accordingly, in one aspect, a system for directing a laser beam to a treatment area includes a solid core chalcogenide glass fiber, where the chalcogenide glass is adapted to minimize transmission losses at wavelengths in a range from about 9 μm up to about 10 μm. The system also includes a CO₂ laser that has a wavelength greater than about 9 μm and less than about 10 μm. The CO₂ laser is optically coupled to the solid core fiber. The system also includes a laser controller for controlling a parameter of the laser beam at least according to a selected treatment.

In some embodiments, the chalcogenide glass includes arsenic and selenium and is further characterized by an absence of tellurium or germanium, or both, thereby minimizing transmission losses of the CO₂ laser. Absence of tellurium or germanium, or both generally means that the material that is absent is not introduced during the manufacturing of the chalcogenide glass or is introduced in trace amounts only, such as about 100 parts-per-million (ppm). In one embodiment, the chalcogenide glass includes about 35 mole percent arsenic and about 65 mole percent selenium, and a trace amount of tellurium. The chalcogenide glass may include either tellurium or germanium, but not both.

In some embodiments, the laser controller is adapted to select a laser pulse repetition rate and/or laser energy per pulse at the treatment area. The laser energy per pulse may be selected by selecting a pulse duration. The treatment area may include a tissue, and the selected treatment may be based on, at least in part, a property of the tissue. For example, the tissue to be treated may include hard tissue and/or soft tissue.

In some embodiments, the system includes a visible spectrum fiber physically but not optically coupled to the chalcogenide glass fiber, and a visible spectrum illumination source optically coupled with the visible spectrum fiber. The system may also include a cooling system for directing one or more of a gas flow and a mist to the treatment area. The chalcogenide glass fiber may be adapted to direct the laser beam to the treatment area through the gas flow and/or the mist.

A chalcogenide fiber can be used to deliver radiation from a laser source to a handpiece, which may then deliver the radiation to a treatment region, and/or from a handpiece to the treatment region. As such, in one embodiment, the system includes a handpiece having an inlet for receiving radiation and a tip for directing the radiation to a treatment area. The solid core chalcogenide glass fiber is optically and physically coupled to the inlet of the handpiece, for delivering radiation transmitted through the solid core chalcogenide glass fiber to the handpiece. Such radiation may be directed to the treatment area through the handpiece tip. In some embodiments, the system includes a handpiece having a tip, and the solid core chalcogenide glass fiber is coupled to the handpiece such that at least a portion of the solid core chalcogenide glass fiber emerges from and extends outside the tip of the handpiece. The laser radiation is transmitted through the solid core chalcogenide fiber and may thus be directed to the treatment region. Another chalcogenide fiber may be used to deliver laser radiation to the handpiece via an inlet thereof.

In another aspect, an apparatus for directing a laser beam to a treatment area includes a CO₂ laser, a solid core chalcogenide glass fiber coupled to the CO₂ laser, and a visible spectrum fiber physically but not optically coupled with the chalcogenide glass fiber. The CO₂ laser may have a wavelength greater than about 9 μm and less than about 10 μm. In one embodiment, the CO₂ laser has a wavelength of about 9.3 μm. The chalcogenide glass may include arsenic and selenium.

In another aspect, an apparatus for transmitting a laser beam to a treatment area includes a solid core chalcogenide glass fiber adapted to direct therethrough a CO₂ laser beam. The apparatus also includes at least one second optical fiber adapted to transmit radiation at a wavelength outside a range of about 9 μm up to about 10 μm. The second fiber is physically but not optically coupled to the chalcogenide glass fiber. In addition, the apparatus includes a hand piece coupled to both the chalcogenide glass fiber and the second optical fiber. The hand piece is adapted to direct the laser beam received via the solid core fiber to the treatment area. The second fiber may include one or more of silica, germanium, sapphire, and a fluoride. The coupling between the chalcogenide fiber and the second fiber may be achieved using one or more of outer-surface bonding, substantially in-parallel bonding on a substrate, co-axial bonding, and encapsulation within an enclosure.

In another aspect, a method of directing a laser beam to a tissue includes directing laser energy from a CO₂ laser, having a wavelength greater than about 9 μm and less than about into a solid core chalcogenide glass fiber optically coupled to the CO₂ laser. The method also includes directing at least a portion of the laser energy to a selected spot of the tissue via a tip of the fiber and through a medium comprising at least one of a gas and a mist. The mist may include air and water.

In some embodiments, the method also includes controlling a parameter of the CO₂ laser based on a property of the tissue, a selected treatment, or both. The parameter that is controlled may a laser pulse repetition rate, laser energy per pulse at about a surface of the tissue, or both. The laser energy per pulse at input of the chalcogenide fiber and/or at about a surface of the tissue can be selected by selecting the duration (i.e., the ON duration) of the laser pulses.

In some embodiments, the method includes directing visible light to the tissue during a treatment thereof via a visible spectrum optical fiber physically but not optically coupled to the solid core fiber. The method may also include directing to the tissue a flow of a medium that includes a gas and/or a mist. At least a portion of the laser energy may be directed to the selected spot through the medium, where the medium does not absorb the laser energy in substance, i.e., less than 0.1%, 0.2%, 0.5%, 1%, 2%, 5%, or 10% energy delivered to the medium is absorbed thereby.

### BRIEF DESCRIPTION OF THE DRAWINGS

Various embodiments of the present version of the invention will be more fully understood with reference to the following Detailed Description in conjunction with the drawings of which:

FIG. 1 schematically shows a composite beam delivery system, for delivery of infrared radiation and radiation of at least one other wavelength, according to one embodiment;

FIG. 2 shows a schematic representation of a laser treatment system that includes a pulsed laser controller, as well as coolant and air delivery systems, according to one embodiment;

FIG. 3 shows a composite beam delivery hand piece tip for directing infrared radiation and a mist and/or gas to a treatment area, according to one embodiment;

FIG. 4 shows a composite laser beam delivery hand piece tip, for directing infrared radiation and radiation of at least one other wavelength to a treatment area, according to one embodiment;

FIGS. 5A and 5B show power output data of an optical fiber and a relationship between the power output and a laser beam parameter, according to one embodiment;

FIGS. 6A-6C show power output data of an optical fiber and a relationship between the power output and the laser beam parameter for which a relationship is depicted in FIG. 5B, according to another embodiment;

FIGS. 7A-7C show power output data of an optical fiber and a relationship between the power output and another laser beam parameter, according to one embodiment; and

FIGS. 8A-8E depicts a CO₂ laser system for medical applications, according to one embodiment.

### DETAILED DESCRIPTION

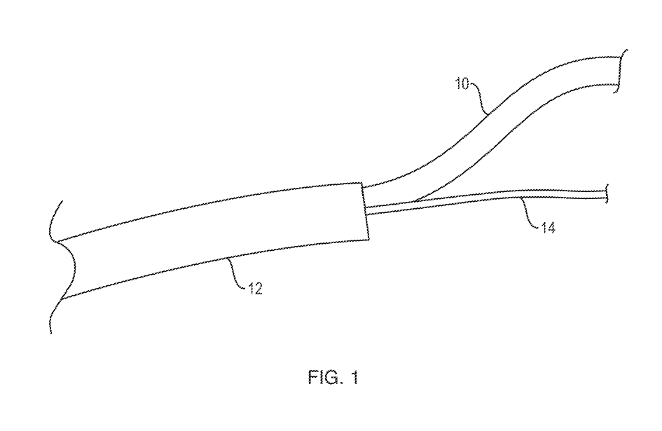

FIG. 1 shows a composite fiber including a chalcogenide optical fiber 10, a furcation tube 12, and a visible-spectrum optical fiber 14. The visible-spectrum optical fiber can be used to transmit radiation at wavelengths not effectively transmitted by the chalcogenide fiber. To this end, the visible-spectrum fiber 14 may include silica, germanium, sapphire, and/or a fluoride, i.e., materials that permit transmission of radiation in the visible spectrum. As such, the fiber 14 may transmit visible light such as the light from a red or green marking/pilot laser. The furcation tubing 12 may be used to jacket or enclose the chalcogenide fiber 10 and the visible-spectrum fiber 14. The furcation tubing may include a composite tube that includes an inner and outer polymer tube and a reinforcing material, such as fiberglass, or metal between the inner and outer tubes. The furcation tubing 12 can protect the optical fibers 10, 14 during use.

In one embodiment, the chalcogenide fiber 10 can be formed as a core/clad fiber. The chalcogenide fiber may include Arsenic (As) and Selenium (Se). The As—Se glass typically has a rate of refractive index change to a change in temperature that is very low, e.g., about 1×10⁻⁶/° C. Generally, the lower the rate of change of refractive index to a change of temperature the more heat the fiber may absorb, before thermal lensing causes the fiber to break. As noted in the background, the use of As—Se glass fibers for delivering radiation from a continuous wave (CW) CO2 laser at 4 and 10 mm wavelengths is known. Many hard and/or soft tissue treatments, however, require the use of a pulsed laser beam at high input power, e.g., at 2 W, 5 W, 15 W, 20 W, 30W, 50W etc. Hard tissue ablation can be achieved at clinically viable rates using a 9.3 μm laser with energy per pulse (pulse energy) of about 10 mJ, 15 mJ, 20 mJ, 30 mJ, etc., and average power of 7 W, 10 W, 15 W, 20 W, 30 W, 50 W, etc. Soft tissue ablation at clinically viable rates can be achieved using a 9.3 μm laser over a wider range of pulse energy not generally in excess of 150 mJ, and usually from about 1W up to about 10 W average power. Typically, the repetition rates for hard tissue ablation are greater than those used for soft tissue ablation.

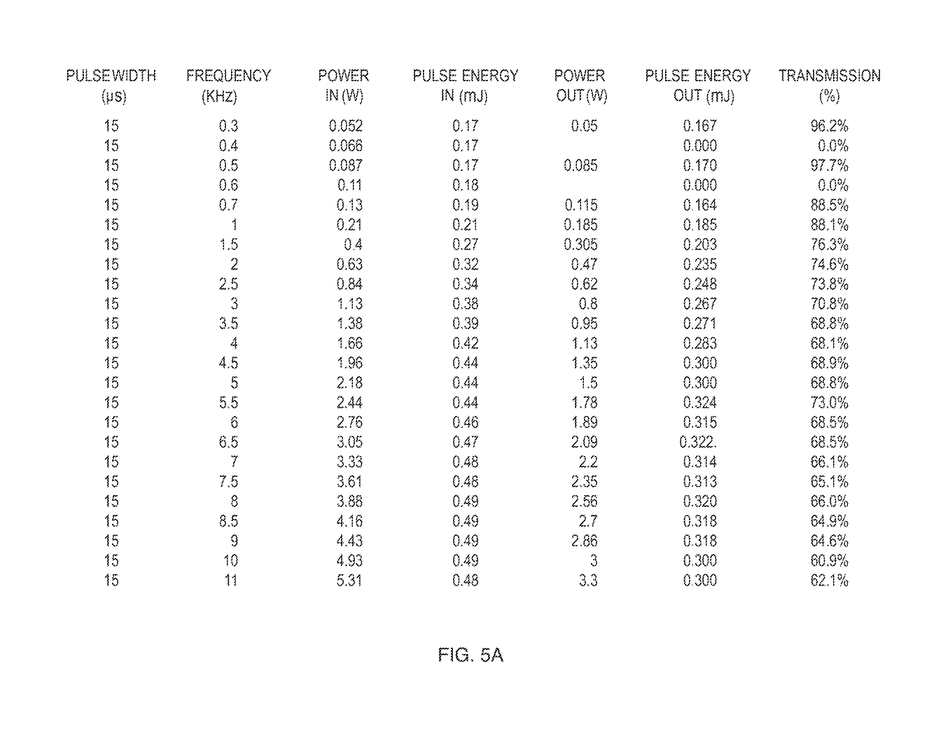

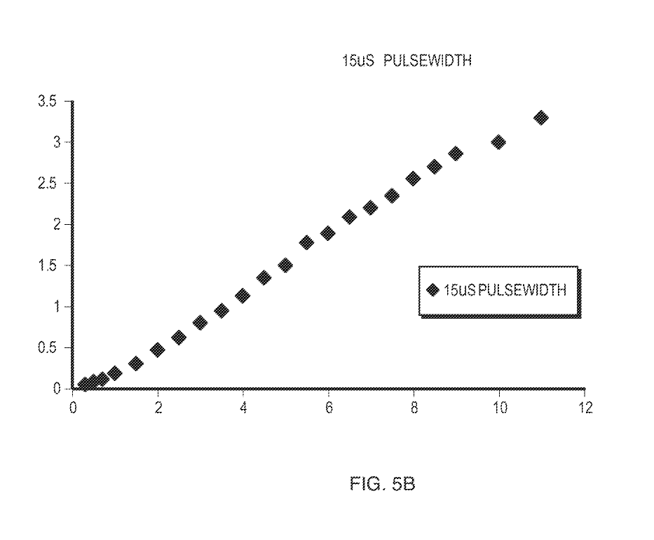

To understand the transmission of a pulsed laser beam at wavelengths in the 9-10 μm range through a chalcogenide fiber, in various experiments, the output power delivered through a chalcogenide fiber was determined by adjusting various laser beam parameters. The results of these experiments are described below with reference to FIGS. 5A-7C. A Coherent C-30 9.3 μm laser was used in the studies. This laser may lack peak power levels high enough to perform ablation of dental enamel at clinically viable rates. This laser, however, can ablate dentin, osseous, and soft tissue at clinically viable rates. More powerful 9. 3 μm lasers, such as one used in Solea™ from Convergent Dental of Natick, Mass., can ablate enamel as well as dentin, osseous tissue, and soft tissue at clinically viable rates. The chalcogenide fiber used in these studies has a composition of about 35% As and 65% Se. The chalcogenide fiber used in these studies was produced without the use of additional purification steps described in U.S. Pat. No 7,418,835 or elsewhere. These studies revealed that the high capacity of the As—Se chalcogenide fiber to absorb heat without failure can enable transmission of radiation from 9-10 μm lasers at high power levels that are suitable for treatment of hard and/or soft tissue. As described below, various parameters of the transmission can be controlled for efficient and reliable delivery of pulsed laser beams.

The purity of the As—Se glass can also contribute to the maximum transmitted power that can be transmitted via a chalcogenide fiber. Containments in the As—Se glass absorb the infrared light and cause the fiber to heat. In one embodiment, the chalcogenide fiber is purified using techniques such as those or similar to those described in U.S. Pat. No. 7,418,835.

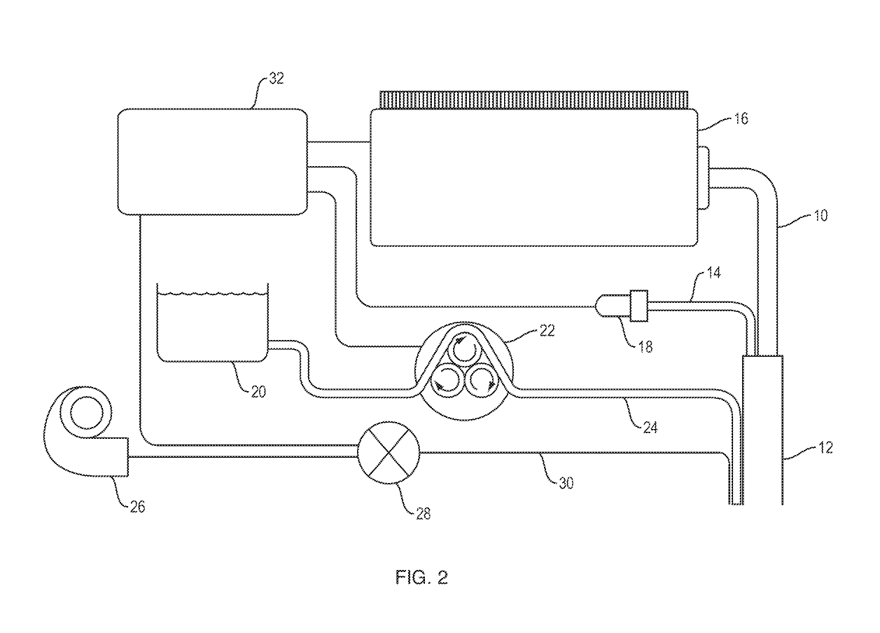

FIG. 2 illustrates a laser system for medical treatment. The system includes a CO₂ laser 16, coupled to the chalcogenide fiber 10. The CO₂ laser may include an isotopic CO₂ laser incorporating an isotope of oxygen with a molecular weight of 18 g/mol. The incorporation of this isotope of oxygen generally results in the CO₂ laser emitting a laser beam at a wavelength of about 9.3 μm. The CO₂ laser 16 can be operated in pulsed mode. The infrared 9.3 μm laser beam is well suited for absorption in many biologic substances, but is invisible to a typical human eye.

A laser diode 18 may therefore be coupled to the visible-spectrum fiber 14. In one embodiment, the laser diode may output a beam in the visible spectrum. The visible-spectrum laser beam may be directed along with the CO₂ laser beam toward an area to be treated. As the two optical fibers 10, 14 are physically (not optically) coupled (e.g., bonded together in a side-by-side manner, co-axially, etc.), a spot upon which a laser beam transmitted through the visible-spectrum fiber 14 can be substantially the same spot upon which the CO₂ laser beam transmitted through the fiber 10 may impinge. Therefore, the beam of the laser diode 18 can be used as a targeting device, allowing an operator to aim the non-visible CO₂ laser beam to a spot and/or an area of treatment.

In various embodiments, the laser diode 18 is selected to emit a wavelength such that the emitted light can be easily seen at or near the treatment area. For example, for treatment of a tissue, the wavelength of the laser diode 18 can be selected such that the visible light due to the emission at or near the tissue surface contrasts with the tissue color. Wavelengths that are generally green such as 532 nm or 520 nm can often be easily seen at or near hard and soft tissue surfaces. As such, a laser diode operating at a generally green wavelength is well suited for targeting a tissue.

In another embodiment, the laser diode 18 is selected to have a wavelength that fluoresces when directed at certain tissue. For example, radiation in a 410-470 nm wavelength range can produce significant differences in intensity and/or spectral profile when directed to a brain tumor relative to when such radiation is directed to generally unaffected brain tissue. The fluorescence of tumors at a specific wavelength may be magnified with the use of certain drugs. In another embodiment, the laser diode 18 emits radiation in a range of about 600 nm up to about 700 nm. In this wavelength band, carious root tissue is distinguishable, by fluorescence spectra, from healthy tooth root tissue.

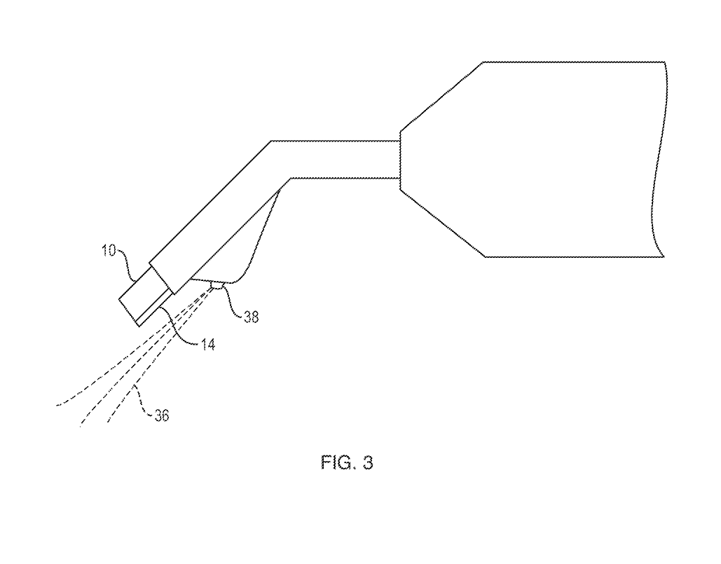

With reference to FIGS. 2 and 3, a coolant source 20 is in fluidic communication with a pump 22. The pump is in fluidic communication with a cooling line 24. The pump 22 can be a peristaltic type pump. The cooling line 24 can deliver the coolant from the coolant source 20 to produce a mist 36 (shown in FIG. 3), directed to the treatment area. The coolant may include distilled water. A source of pressurized air 26, such as an air compressor or tank, may be included in the system or may be provided externally. The air pressure source 26 is in fluidic communication with a valve 28, e.g., a solenoid type valve. The valve 28 allows for flow of compressed air from the compressed air source 26 to be controlled. The valve is in fluidic communication with an air-line 30, that can be used to supply an air flow so as to produce the mist 36 (shown in FIG. 3), that is directed to the treatment area.

FIG. 2 also shows a controller 32 that can control the pulsed operation of the CO₂ laser 16 or other laser beam sources. The controller 32 can be a processor running a software application and/or may include custom and/or reconfigurable circuitry implemented using, e.g., application specific integrated circuits (ASICs), field programmable gate arrays (FPGAs), etc. The pulsed operation of the laser may be defined by the repetition rate and/or duration of the pulses. In some embodiments, a pulse duration of the laser is related to the thermal time constant of the material being treated. For example, the thermal time constant for dental enamel is about 2 μs and, as such, a suitable pulse duration for ablating enamel without damaging the surrounding tissue can be about 20 μs. Larger pulse durations can be used, however, without overly heating surrounding tissue, using a coolant such as an air water mist. The CO₂ laser may be pulsed at high repetition rates, in excess of 10 kHz. The controller 32 may also control the initiation of the laser diode 18 and the valve 28. The controller 32 may also control the pump 22, so as to control the rate of flow of the coolant. The controller 32 may also control the valve 28, so as to control flow of compressed air.

FIG. 3 shows a hand piece tip that can supply a cooling mist 36. The chalcogenide fiber 10 and the visible-spectrum fiber 14 may extend out from the hand piece tip. The mist 36 can be produced via an orifice 38. The coolant line 14 and the air-line 24 (shown in FIG. 2) can provide the coolant and air, respectively, to the orifice 38 so as to produce the mist 36. One or more mist orifices may be used to provide adequate cooling mist to the treatment area if mist is desired for treatment. In some embodiments, a flow of pressurized gas (e.g., air) may be directed to the treatment area using the hand piece tip, instead of using the mist. In other embodiments, a second flow of pressurized gas (e.g., air) may be used in addition to the mist. These gas flows can facilitate cooling and/or cleansing of the treatment area. Generally, the delivery of the laser beam through the gas flow and/or mist is substantially unaffected, i.e., only a minimal portion, if any, of the laser beam energy is absorbed in the gas and/or mist, such that substantially all of the laser energy in the chalcogenide fiber 10 is delivered to the treatment area for effective treatment.

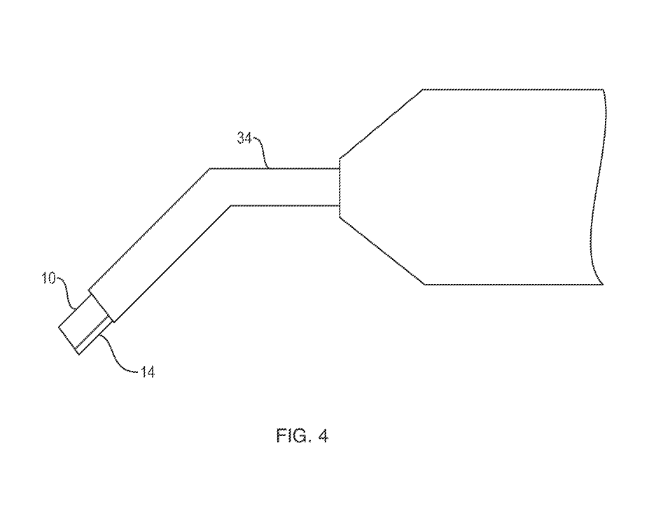

A hand piece tip depicted in FIG. 4 includes a cannula 34. In one embodiment, the chalcogenide fiber 10 and the visible-spectrum fiber 14 are routed through the cannula 34 and can emerge from an outlet of the tip. The cannula can be constructed using metals, such as stainless steel and/or nickel; polymers, such as polyether ether ketone (PEEK); or a combination of the two. The cannula 34 may be bent, as depicted in FIG. 4, or may be straight, in order to direct the chalcogenide fiber 10 and, thus, the laser beam emitted there through, toward the treatment area. The chalcogenide and visible-spectrum fibers can extend outside the tip of the cannula 34 by some amount, typically between 2-10 mm, depending on the accessibility of the treatment area. In various embodiments, the length of the chalcogenide fiber 10 extending from the outlet of the cannula 34 and/or the length of the visible-spectrum fiber 14 extending from the outlet of the cannula 34 are selected such that the respective tips of the fibers 10, 14 are not in contact with the tissue to be treated. During treatment, the distance between the tip of fiber 10 and/or tip of fiber 14 can be maintained at about 2 mm, 3 mm, 5 mm, etc. The chalcogenide fiber 10 and the visible-spectrum fiber 14 may be held together by the cannula 34 or may be bonded together. The bonding may be achieved through adhesives, such as cyanoacrylate, or by fusing the glass claddings together.

In order to deliver laser energy to the treatment region in an effective manner, i.e., to heat the tissue to be treated without damaging the optical fiber, the tissue to be treated (unless it is to be ablated), and the surrounding tissue, while avoiding excessive treatment time, the controller 32 (shown in FIG. 2) can control various parameters of the laser beam. For example, with reference to FIGS. 5A and 5B, the pulse duration (i.e., the ON time of a pulse) can be adjusted to a selected value, e.g., 15 μs, and the pulse frequency can be varied within a specified range, e.g., from 0.3 kHz up to 10 kHz. Studies using a Coherent C-30 9.3 μm CO₂ laser and a As—Se chalcogenide fiber having a composition of about 35% As and about 65% Se were performed. Results from these studies are included in FIGS. 5-7. Radiation from the Coherent C-30 9.3 μm CO₂ laser was delivered through the As—Se chalcogenide fiber (e.g., the fiber 10 shown in FIG. 1), and the output power is measured, as shown in FIG. 5A. FIG. 5B shows that the relationship between the output power and the pulse repetition rate/frequency is generally linear for the selected pulse duration of 15 μs.

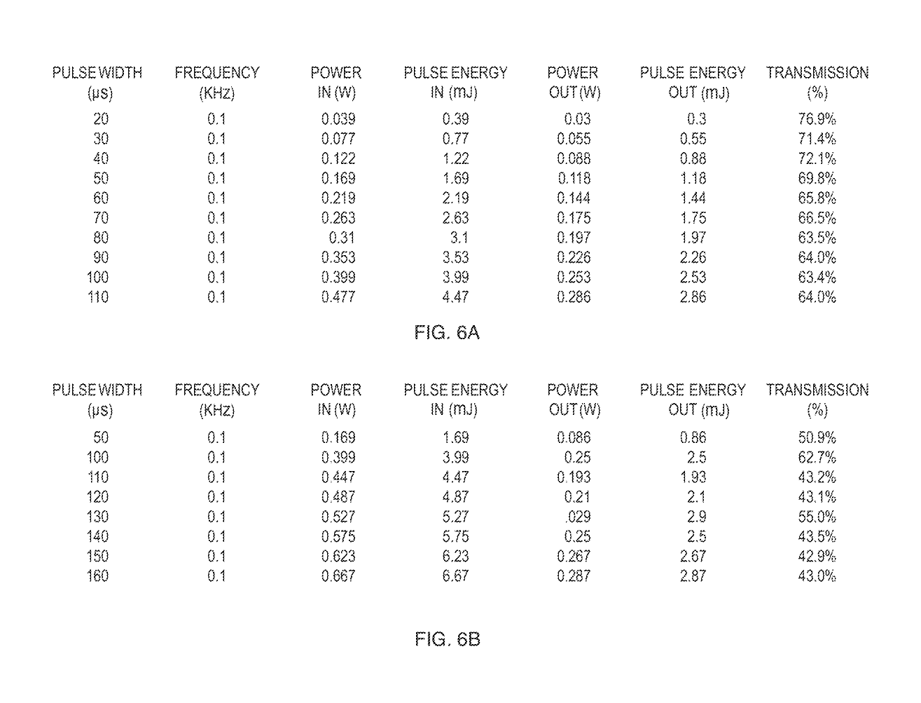

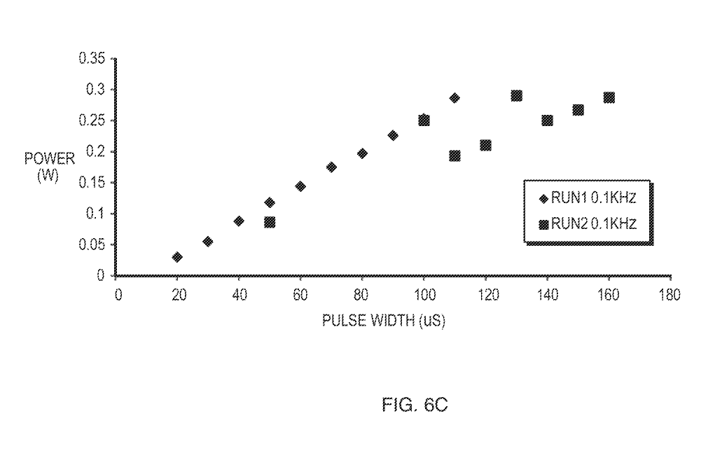

It should be understood that the values of the parameters described above are illustrative only, and in various embodiments, the pulse duration can range from about 1 μs up to about 500 μs or up to about 1 ms. The pulse repetition rate can range from 50 Hz up to about 20 kHz. FIGS. 6A and 6B, for example, show a table of output power for a different setting of pulse duration of 40 μs. The frequency is varied from 0.1 kHz up to 3 kHz in RUN1, and from 3 kHz up to 7.2 kHz in RUN 2. FIG. 6C shows that the relationship between the output power and the pulse repetition rate/frequency is generally linear for the selected pulse duration of 40 μs.

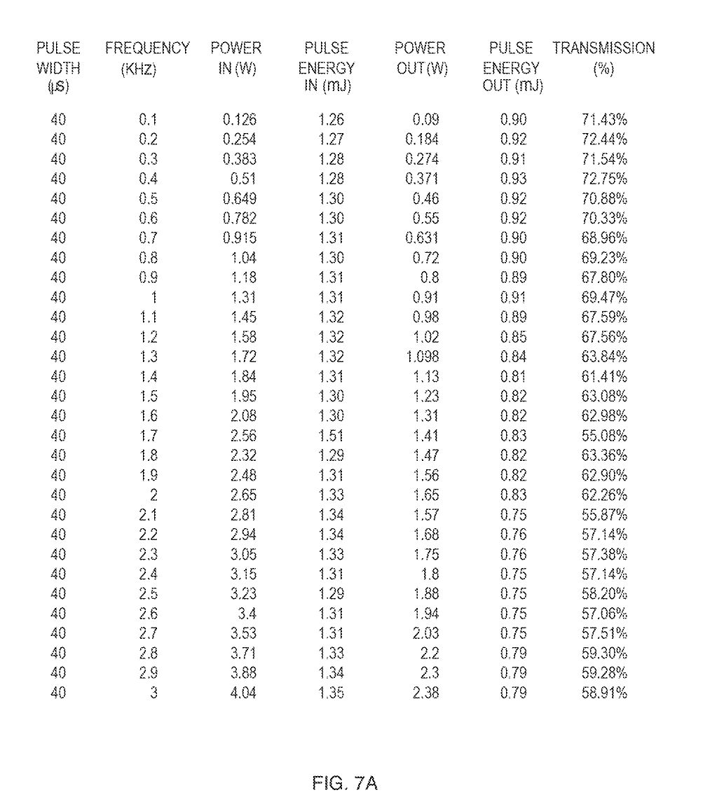

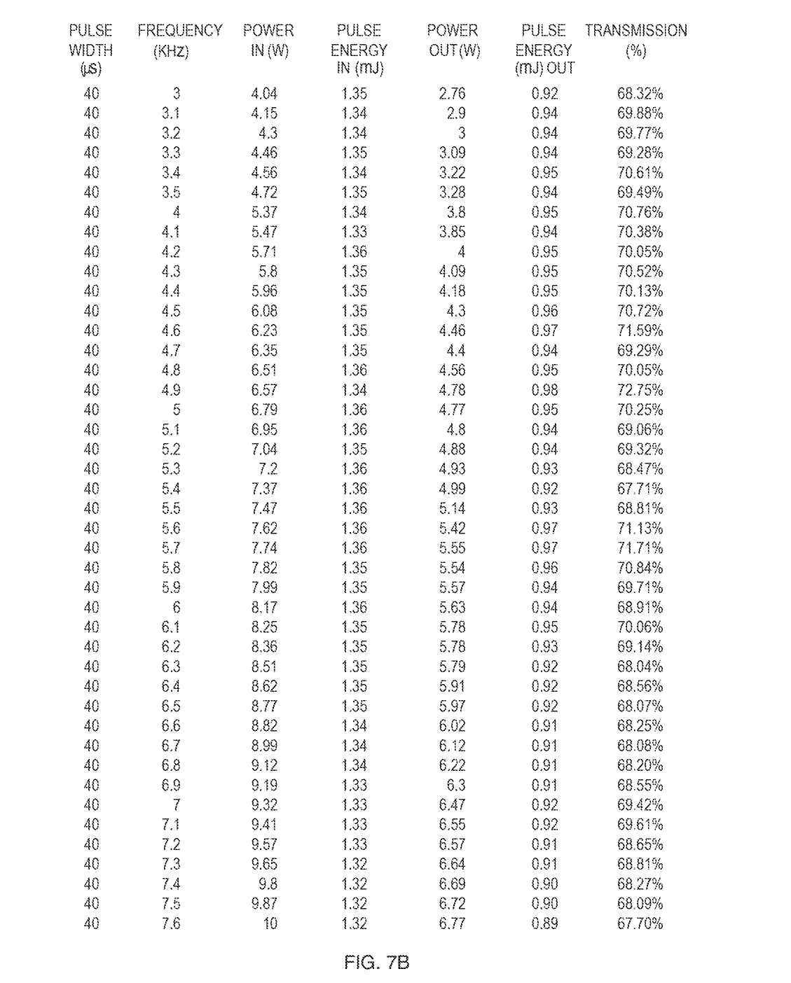

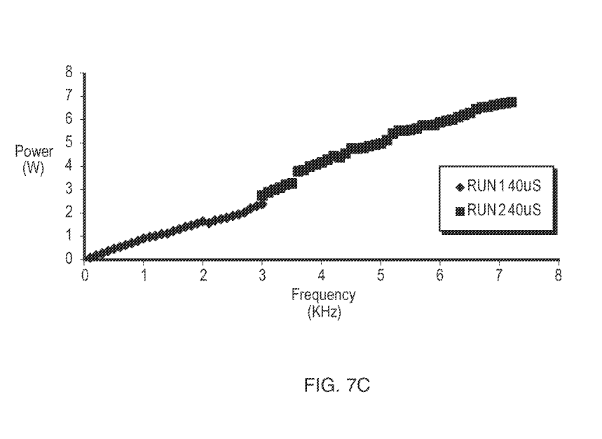

With reference to FIGS. 7A and 5B, the pulse repetition rate/frequency can be adjusted to a selected value, e.g., 0.1 kHz, and the pulse duration can be varied within a specified range, e.g., from 20 μs up to 110 μs as shown in FIG. 7A, or from 50 μs up to 160 μs as shown in FIG. 7B. The pulse duration generally corresponds to the energy per pulse. Radiation from the Coherent 9.3 μm CO₂ laser using these parameters is delivered through the As—Se chalcogenide fiber (e.g., the fiber 10 shown in FIG. 1), and the output power is measured, as shown in FIGS. 7A and 7B. FIG. 7C shows that the relationship between the output power and the pulse duration is generally linear for the selected pulse frequency of 0.1 kHz.

In some embodiments, output power data are collected for a number of different pulse durations and ranges of pulse repetition rates, and respective relationships there between are determined. Output power data are also collected for a number of pulse repletion rates and ranges of pulse durations, and respective relationship there between are determined. Such information is supplied to a controller (e.g., the controller 32 shown in FIG. 2). Output power data can also be collected by setting the input power to different values, for different types of lasers, e.g.,9.6 μm CO₂ laser or high powered 9.3 μm CO₂ laser, such as one used in Solea™ from Convergent Dental of Natick, Mass., and for different types of chalcogenide fibers, such as As—Se fibers produced with high purity using methods such as those or similar to those described in U.S. Pat, No. 7,418,835, and such data may be supplied to the controller 32, as well.

A particular combination of the pulse duration and repetition rate determines, at least in part, the energy per second or laser power transmitted through and delivered to the treatment region. The power required at the treatment region may depend on various factors including the nature of the tissue (e.g., whether the tissue is hard or soft), condition of the tissue, the nature of the treatment (e.g., ablation, de-carbonation, caries detection, caries prevention, etc.), type of coolant used (e.g., mist or airflow), etc. An operator may select a required power for a particular treatment. The laser beam generator generally determines some parameters of the laser beam to be used for treatment. For example, the laser beam generator may determine a range of available pulse widths, a range of available repetition rates, and a range of the amount of power that can be input to the optical fiber-based beam delivery system, such as the power that can be supplied to the chalcogenide fiber 10 shown in FIG. 1.

Using the available laser beam parameters and the required power output, the controller 32 shown in FIG. 2 may select a particular combination of pulse width and frequency. For example, to deliver about 1.5 W power, the controller may select a pulse width of 15 μs and a pulse repetition rate of 5 kHz. These parameters are specifically selected for the Coherent C-30 9.3 μm CO₂ laser that is optically coupled to the As—Se chalcogenide fiber 10 shown in FIG. 1 and used in the studies described in FIG. 5-FIG. 7. If the same laser is used with a different beam delivery system, such as a hollow waveguide system, the combination of pulse width and repetition rate described above may not yield the required output power of approximately 1.5 W. Similarly, if radiation from a 10.6 μm CO₂ laser is delivered through the As—Se chalcogenide fiber 10, to obtain the required output power of 1.5 W, a different combination of pulse width and pulse repetition rate may be required. In general, a laser, as a source of radiation, is characterized by the laser's power level, wavelength, and, optionally, pulsing characteristics. As such, even if radiation from two 9.3 μm CO₂ lasers is delivered through a As—Se chalcogenide fiber, if the power, pulsing, and/or any other characteristic of the two 9.3 μm CO₂ lasers are different, to obtain a specified output power, the combination of pulse width and pulse repetition rate required for one laser can be different from the combination of pulse width and pulse repetition rate required for the other laser. As such, for use by a controller, data such that described above may need to be collected for a particular laser and a particular chalcogenide fiber used to transmit radiation from the laser.

The controller 32 shown in FIG. 2 may use a relationship between the output power and pulse frequency/repletion rate (e.g., the relationships depicted in FIGS. 5B and 6C), for selecting a suitable repetition rate. For example, if the operator chooses to change the required output power from 1.5 W to 1 W, the controller 32 can determine that the pulse repetition rate should be adjusted to 3.5 kHz. The laser beam generator may not supply pulses at 3.5 kHz, however, and may permit changes in steps of 1 kHz only. Using the generally linear relationship, the controller 32 may then select a repetition rate of 3 kHz. The controller 32 may also signal the operator that the output power would be about 0.8 W, i.e., less than the required amount of 1 W.

In some instances, the operator can tolerate more than the required output power, e.g., not to increase the treatment time, if the operator also determines that the excess output power would not be harmful. Therefore, in some embodiments, in response to a signal from the operator, the controller 32 may suggest adjusting and/or adjust the pulse repletion rate to 4 kHz, and signal the operator that the output power is about 1.13 W. In some embodiments, in addition to providing the indication that the required power cannot be delivered, the controller 32 may also suggest a change in the pulse duration. With reference to FIGS. 6A-6C, for example, the controller may suggest adjusting the pulse width to 40 μs and adjusting the pulse repetition rate to 1 kHz, so that the power output is about 0.91 W, which may be preferable to the 0.8 W power output that can be achieved using the 15 ms pulse width, 3 kHZ repetition rate combination. In general, the controller can adjust the repetition rate, pulse duration, or both using available data.

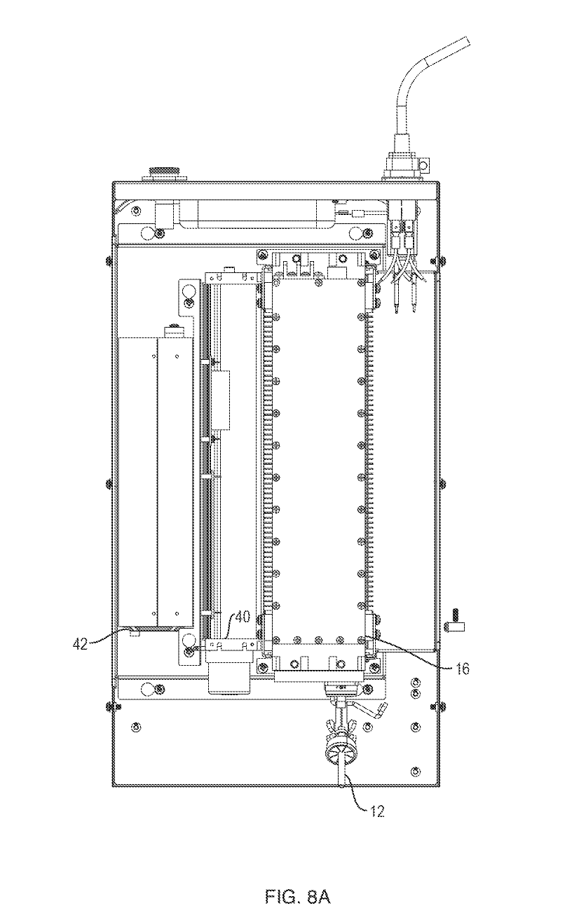

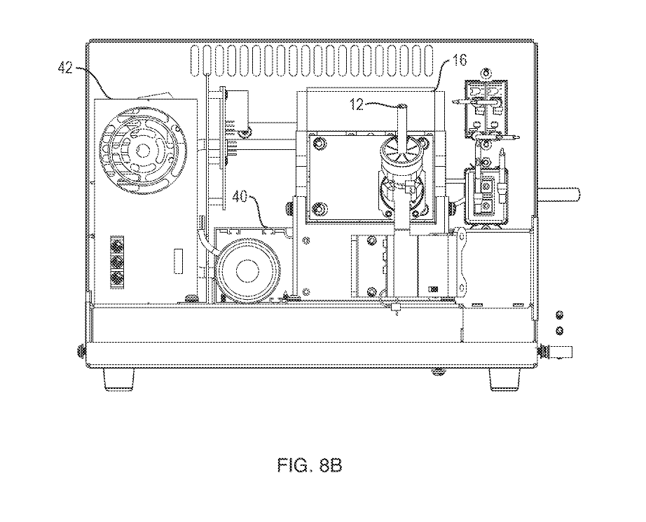

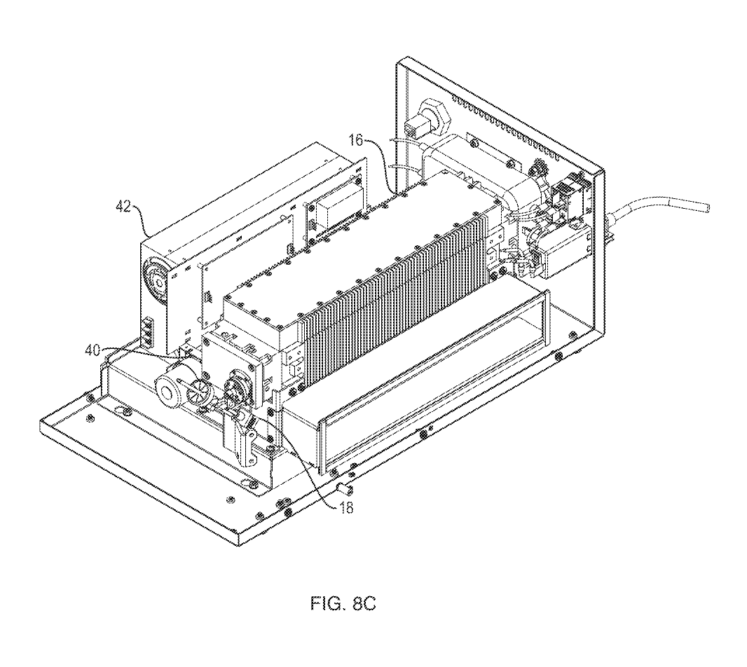

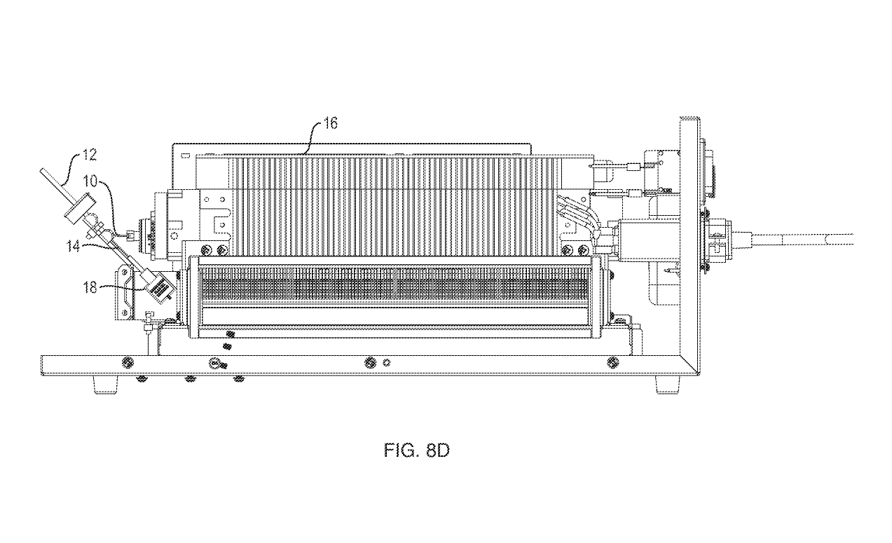

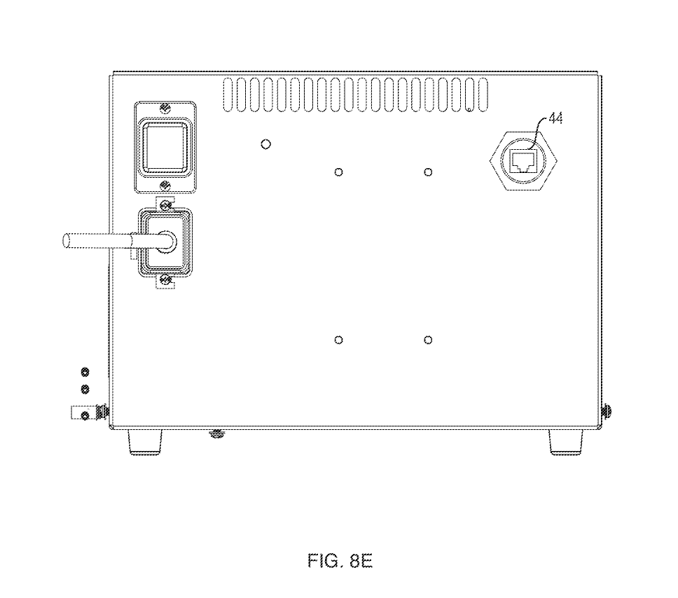

An example layout of a medical laser treatment system is shown in FIGS. 8A-8E. The CO₂ laser 16 can be an air cooled 30 W laser, such as the Coherent C-30 9.3 μm CO₂ laser that is optically coupled to the chalcogenide fiber 10. As such, the laser beam output from the laser 16 may be transmitted through the chalcogenide fiber 10. An example chalcogenide fiber has a diameter of 200 μm and 250 μm for the core and cladding, respectively. In another example, a chalcogenide fiber has diameters of 300 μm, 370 μm, and 550 μm, for the core, clad, and buffer, respectively. This fiber uses a single layer acrylate for the buffer.

An example laser diode 18 is a green 520 nm diode, which is coupled to a single mode visible-spectrum fiber 14. Multi-mode fibers may also be used in some embodiments. An example visible-spectrum fiber has a diameter of 3.9 μm, 125 μm, and 245 μm for the core, cladding, and coating, respectively. The furcation tubing 12 can be a Hytrel tubing with a 900 μm outside diameter. The cooling for the CO₂ laser 16 may be provided using an axial blade fan 40. The CO₂ laser, diode laser, and axial blade fan may be powered with a DC power supply 42. An external laser controller may be used to operate the laser system. The external laser controller may receive power output data such as that described with reference to FIGS. 5A-7C. The external laser controller can be connected with the laser system through an RJ45 jack 44, allowing the external laser controller to control the pulsed operation of the CO₂ laser.

A cooling mist is not generally required for the ablation of soft tissue. A soft tissue treatment procedure typically involves an operator holding a hand piece attached to the distal ends of the chalcogenide and visible fibers. The hand piece tip, (e.g., the tip shown in FIG. 4), may be located at the distal end of the hand piece. The operator may bend the cannula of the hand piece tip in order to provide access to the area to be treated. The tip of the visible fiber may be illuminated using a visible spectrum laser. The operator may set the repetition rate and pulse duration for the CO₂ laser, e.g., according to the treatment selected and/or the type and/or condition of the tissue. In some embodiments, the operator may employ the external controller to suggest and/or select one or more of the repetition rate, the pulse duration, and the input power. The light transmitted through the visible-spectrum fiber can allow the operator to identify locations (e.g., spots) on the tissue that are to be treated by the CO₂ laser, when it is pulsed. The operator may then locate the hand piece tip near the area to be treated such that the chalcogenide fiber may contact the area to be treated or may be at a small distance (e.g., a few mm) from the area to be treated. The operator may then activate the CO₂ laser at the selected repetition rate and pulse duration, to treat the area and/or the identified spots thereon.

Hard tissue procedures, such as the ablation of osseous tissue and teeth, can be performed in a similar manner. Typically, hard tissue procedures additionally require the use of a coolant in order to prevent carbonization. Therefore, mist or flow of a gas may be directed to the treatment area. A hand piece tip with a mist orifice (e.g., the tip shown in FIG. 3), may be used to provide a cooling mist. The operator, the external controller, and/or another controller may set the coolant flow rate, air pressure, and/or any other mist settings prior to commencing the procedure. The cooling mist is typically supplied when the CO₂ laser is activated. To this end, the external controller or the other controller can initiate coolant and/or gas flows just prior to activating the CO₂ laser, which may be activated in response to a command from the operator, for example by stepping on a foot pedal. Applicant's co-pending U.S. Patent Application No. 13/894,067, entitled “Apparatus and Method for Controlled Fluid Cooling During Laser Based Dental Treatments,” filed on May 14, 2013, and published as US2013/0323672A1 on December 5, 2013 describes a coolant and a mist system and a controller therefor, and is incorporated herein by reference in its entirety. Applicant's co-pending International (PCT) Patent Application No. PCT/US2014/014674, entitled “Dental Laser Apparatus and Method of Use with Interchangeable Hand Piece and Variable Foot Pedal,” filed on Feb. 4, 2014, describes various hand pieces for delivering a laser beam to a treatment area, and is incorporated herein by reference in its entirety, as well.

A carbonate removal procedure for caries prevention generally does not require the use of a mist. The pulse duration and repetition rate of the CO₂ laser may be suitably set for this procedure by the operator and/or by a controller, as described above. The operator can direct the hand piece tip toward the tooth surface and may activate the CO₂ laser. The entire surface of the tooth or at least a significant portion thereof may be irradiated by moving the hand piece to direct the beam to different areas on the tooth. This procedure can be repeated for one or more other teeth.

Having described herein illustrative embodiments and best mode of the present invention, persons of ordinary skill in the art will appreciate various other features and advantages of the invention apart from those specifically described above. It should therefore be understood that the foregoing is only illustrative of the principles of the invention, and that various modifications and additions can be made by those skilled in the art without departing from the spirit and scope of the invention. Accordingly, the appended claims shall not be limited by the particular features that have been shown and described, but shall be construed also to cover any obvious modifications and equivalents thereof.

What is claimed is:

1. A system for directing a laser beam towards a treatment area, the system comprising:
a solid core chalcogenide glass fiber, the chalcogenide glass being adapted to minimize transmission losses at wavelengths in a range from about 9 μm up to about 10 μm;
a CO₂ laser that: (i) has a wavelength greater than about 9 μm and less than about 10 μm, and (ii) is coupled to the solid core fiber; and
a laser controller for controlling a parameter of the laser beam according to a selected treatment.

2. The system of claim 1, wherein the chalcogenide glass comprises arsenic and selenium and is further characterized by an absence of at least one of tellurium and germanium, thereby minimizing transmission losses of the CO₂ laser.

3. The system of claim 1, wherein the chalcogenide glass comprises about 35 mole percent arsenic and about 65 mole percent selenium, and a trace amount of tellurium.

4. The system of claim 1, wherein the chalcogenide glass comprises one of tellurium and germanium.

5. The system of claim 1, wherein the laser controller is adapted to select at least one of a laser pulse repetition rate and laser energy per pulse at the treatment area.

6. The system of claim 1, wherein:
the treatment area comprises a tissue; and
the selected treatment is based, at least in part, on a property of the tissue.

7. The system of claim 6, wherein the tissue comprises at least one of a hard tissue and a soft tissue.

8. The system of claim 1, further comprising:
a visible spectrum fiber coupled to the chalcogenide glass fiber; and
a visible spectrum illumination source coupled with the visible spectrum fiber.

9. The system of claim 1, further comprising:
a cooling system for directing at least one of a gas flow and a mist to the treatment area,
wherein the chalcogenide glass fiber is adapted to direct the laser beam to the treatment area through the at least one of the gas flow and the mist.

10. The system of claim 1, further comprising:
a handpiece having an inlet for receiving radiation and a tip for directing the radiation to a treatment area, the solid core chalcogenide glass fiber being optically and physically coupled to the inlet of the handpiece, for delivering radiation transmitted through the solid core chalcogenide glass fiber to the handpiece.

11. The system of claim 1, further comprising a handpiece having a tip, the solid core chalcogenide glass fiber being coupled to the handpiece such that at least a portion of the solid core chalcogenide glass fiber emerges from and extends outside the tip of the handpiece.

12. An apparatus for directing a laser beam towards a treatment area, the apparatus comprising:
a CO₂ laser;
a solid core chalcogenide glass fiber coupled to the CO₂ laser; and
a visible spectrum fiber coupled with the chalcogenide glass fiber.

13. The apparatus of claim 12, wherein the CO₂ laser has a wavelength greater than about 9 μm and less than about 10 μm.

14. The apparatus of claim 12, wherein the CO₂ laser has a wavelength of about 9.3 μm.

15. The apparatus of claim 12, wherein the chalcogenide glass comprises arsenic and selenium.

16. An apparatus for transmitting a laser beam towards a treatment area, the apparatus comprising:
a solid core chalcogenide glass fiber adapted to direct therethrough a CO₂ laser beam;
at least one second optical fiber adapted to transmit radiation at a wavelength outside a range of about 9 μm up to about 10 μm, the second fiber being coupled to the chalcogenide glass fiber; and
a hand piece coupled to both the chalcogenide glass fiber and the second optical fiber, the hand piece being adapted to direct the laser beam received via the solid core fiber to the treatment area.

17. The apparatus of claim 16, wherein the second fiber comprises at least one of silica, germanium, sapphire, and a fluoride.

18. The apparatus of claim 16, wherein a coupling between the chalcogenide fiber and the second fiber comprises at least one of outer-surface bonding, substantially in-parallel bonding on a substrate, co-axial bonding, and encapsulation within an enclosure.

19. A method of directing a laser beam to a tissue, the method comprising:
directing laser energy from a CO₂ laser, having a wavelength greater than about 9 μm and less than about 10 μm, into a solid core chalcogenide glass fiber coupled to the CO₂ laser; and
directing at least a portion of the laser energy to a selected spot of the tissue via a tip of the fiber and through a medium comprising at least one of a gas and a mist.

20. The method of claim 19, wherein the mist comprises air and water.

21. The method of claim 19, further comprising controlling a parameter of the CO₂ laser based on at least one of a property of the tissue and a selected treatment.

22. The method of claim 21, wherein the parameter is selected from the group consisting of a laser pulse repetition rate and laser energy per pulse at about a surface of the tissue.

23. The method of claim 19, further comprising directing visible light to the tissue during a treatment thereof via a visible spectrum optical fiber coupled to the solid core fiber.

24. The method of claim 19, further comprising:
directing a flow of a medium comprising at least one of a gas and a mist to the tissue,
wherein the at least a portion of the laser energy is directed to the selected spot through the medium.
